(19) United States
(12) Patent Application Publication
(10) Pub. No.: US 2023/0078643 A1
(43) Pub. Date: Mar. 16, 2023

(54) Artificial Muscle of Electrothermally Active Contractile Polymers Device and Method of Manufacturing the Same

(71) Applicants: Dharshika Kongahage, Horsley (AU); Arjang Ruhparwar, Essen (DE); Javad Foroughi, Mount Pleasant (AU)

(72) Inventors: Dharshika Kongahage, Horsley (AU); Arjang Ruhparwar, Essen (DE); Javad Foroughi, Mount Pleasant (AU)

(73) Assignee: Cardiac Innovations, LLC, Deephaven, MN (US)

(21) Appl. No.: 17/943,122

(22) Filed: Sep. 12, 2022

Related U.S. Application Data
(60) Provisional application No. 63/243,170, filed on Sep. 12, 2021.

(51) Int. Cl. A61F 2/50; A61F 2/48

(52) U.S. Cl. CPC A61F 2/50; A61F 2/482; A61F 2002/5066; A61B 2017/00871

(57) ABSTRACT

Artificial muscle device and method of manufacturing the same for the treatment or control of an organ such as the heart. The artificial muscle device comprises artificial polymer actuators or fibers that can work together to form the artificial muscle structure. The artificial fibers are electrothermally active contractile polymers capable of various characteristics, including increased contractile forces. The artificial actuators are knittable or weavable into patterns and shapes to create unique artificial muscles that can be shaped into implantable devices.

### PRIORITY

This application claims the priority benefit of U.S. Provisional Application No. 63/243,170, filed on Sep. 12, 2021, which is hereby incorporated herein by reference in its entirety.

### FIELD

The present invention relates to an artificial muscle device for controlling a tissue or organ and more particularly to an artificial muscle device composed of one or more electrothermally active contractile polymers that are able to control tissues, organs, bones and the like.

### BACKGROUND

Among all transplantation surgeries, heart transplantation is amongst the major surgical procedures of organ replacement. There are approximately 5,000 heart transplantations performed each year worldwide. Furthermore, there are 250,000 deaths reported annually in the United States with 9,00,000 hospitalizations due to heart failure. In contrast, it has been estimated that over 60,000 patients in the United States can benefit from heart transplantation. However, the critical shortage of this organ has led many researchers to explore ways to assist heart patients, at least temporarily, until a transplantation takes place.

In the 1960's researchers developed ventricular assist devices (“VADs”). VADs are mechanical circulatory systems that have clinically evolved as a promising substitute for the treatment of end-stage heart failure due to shortage of donor hearts. VADs reported to date use electric, pneumatic, hydraulic, electromagnetic, and electro-hydraulic systems. VADs having continuous-flow technology has also entered the market.

VADs, however, fall short of an ideal solution for numerous reasons. For instance, VADs use rigid components and/or bulky power systems made from metals or plastic, which are unsuitable for use in some heart patients due to the size limitations. Another shortcoming of conventional VADs is that they have been associated with driveline infections with their external energy source. Additionally, the non-pulsatile blood flow in left VADs has led to new complications such as increased aortic valve regurgitation and spontaneous bleedings.

In the late 1980's and early 1990s surgeons attempted to overcome the shortcomings of the VADs by creating simulated cardiac tissue by placing skeletal muscle around a diseased heart. The skeletal muscle acted as a “backstop” to mechanically assist the diseased cardiac tissue. This approach to solving the problem was called skeletal cardiomyoplasty. However, skeletal muscles as a contractile substrate proved not suitable for long-standing cardiac support.

More recently, researchers have devised regenerative treatment strategies for partial or complete replacement of diseased myocardium. These strategies include cellular cardiomyoplasty which attempts to engineer artificial heart tissue from a mixture of a collagen matrix and neonatal cardiomyocytes. These engineered artificial heart tissues have been transplanted into small animals resulting in some improvement of ventricular function.

However, the engineered artificial heart tissues have several challenges that have slowed down the progress in this field. For instance, finding the optimal cell source remains a challenge as fetal and neonatal cardiomyocytes are not available in large numbers. Additionally, the myocardium is more complex and heterogeneous than other cardiac structures such as heart valves where first tissue engineered products have been introduced to the market. Therefore, production of cells for largescale therapeutic measures is continuing to be a challenge.

More recently, researchers have developed various purely artificial heart tissue devices and systems to overcome the failings of the above cardiomyoplasty devices. In this respect, the purely artificial tissue or muscles constitute a modern alternative for cardiomyoplasty.

These purely artificial heart tissue devices have taken many forms including, patches attached to the cardiac tissue as a treatment for patients with end stage heart failure and pneumatic sleeves placed around the heart that are able to compress or squeeze the heart. The patches fail to provide an effective solution due to their small surface area. Pneumatic systems also have several disadvantages when compared to applicant's invention. For example, they generally require installation of air-producing equipment, which is subject to air leakage. Additionally, they easily develop condensation.

There has been a need for miniaturization and technical advances in material science to develop a more modern approach that address the failings of the conventional cardiac disease treatments.

There is a need for electrically active contractile polymers that can generate significantly higher forces with no fatigue.

There is a need for electrically active contractile polymers that can generate significantly higher forces with no fatigue that can be connected to bone for the purpose of enabling or assisting mobility.

There is a need for electrically active contractile polymers that can augment the heart ventricle and its functions.

There is a need for an electrically active contractile polymer that eliminates the use of anticoagulants.

There is a need for electrically active contractile polymers that can be manufactured by additive manufacturing and that are able to fabricate soft heart chambers with heat insulating polymers.

There is a need for electrically active contractile polymers that incorporate phase change materials for heat-absorbing and printed actuators that can also have an added advantage for the fabrication of AHM.

There is a need for electrically active contractile polymers that can be formed into an artificial muscle that can be implanted into humans and animals.

There is a need for electrically active contractile polymers that can be formed into an artificial muscle that may provide an improved power system for VADs by miniaturization and for maintaining pulsatility.

There is a need for electrically active contractile polymer-based actuators to be used in artificial heart muscle (AHM) as an effective cardiac pouch, sleeve, or patch that are able to avoid direct contact with a patient's blood.

There is a need for miniaturization, speed modulation, total implantability, and preservation of pulsatility with no contact with a patient's blood.

There is a need for novel electrically active contractile polymers that form an apparatus, device, system or part of a method of treatment that may be adapted to various and unique anatomy so that therapy may be properly applied to treat various conditions.

There is a need for novel electrically active contractile polymers that form an apparatus, device, system or part of a method of treatment that may be adapted to any part of a patient's circulatory system, including but not limited to arteries and veins to treat various circulatory disorders.

There is a need for novel electrically active contractile polymers that form an apparatus, device, system or part of a method of treatment that may be adapted to a patient's bladder to treat various urinary or excretory disorders.

The object of the present invention is to address one or more of the above problems, while maintaining the advantages of prior art.

### SUMMARY OF THE INVENTION

Artificial muscle device and method of manufacturing the same for the treatment or control of an organ such as the heart. The artificial muscle device comprises artificial actuators or fibers that can work together to form the artificial muscle. The artificial fibers or actuators are electrically active and more particularly electrothermally active contractile polymers capable of various characteristics, including increased contractile forces. The artificial actuators or fibers are knittable or weavable into patterns and shapes to create unique artificial muscles that can be shaped into implantable devices.

Additional features and embodiments will be apparent from the detailed description and the attached patent claims.

### BRIEF DESCRIPTION OF THE DRAWINGS

The invention will be described in more detail in the detailed description below with reference to the appended drawings, in which:

FIG. 1 is a perspective view of an artificial human muscle device in a sleeve configuration positioned on various organs;

FIG. 2 is a perspective view of an artificial human muscle device in a sleeve configuration positioned on a heart;

FIG. 3 is a perspective view of an artificial human muscle device in a patch configuration positioned on various organs;

FIG. 4A is a perspective view of an artificial human muscle device in a patch configuration positioned on a heart;

FIG. 4B is a perspective view of an artificial human muscle device coupled to and controlling a pump of a VACD;

FIG. 5A illustrates an artificial human muscle device comprised of electrothermally active contractile polymers or fibers configured as a parallel construct;

FIG. 5B illustrates an artificial human muscle device comprised of electrothermally active contractile polymers or fibers configured as a braided construct;

FIG. 6A illustrates an example electrothermally active contractile polymer in a coil configuration;

FIG. 6B an example insulated electrothermally active contractile polymer in a coil configuration;

FIG. 7 is an example coiling process for creating active contractile polymers of artificial muscles;

FIGS. 8A-8D are illustrating an example coating process and method;

FIGS. 9A-9D are examples of hybrid electrothermally active contractile polymers;

FIG. 10 is an example of a single electrothermally active contractile polymer;

FIG. 11 is a table of different hybrid electrothermally active contractile polymers;

FIG. 12A-12B illustrate various hybrid electrothermally active contractile polymers constructions;

FIGS. 13A-13B illustrate various hybrid electrothermally active contractile polymers;

FIG. 14 is a table illustrating physical characteristics of various hybrid electrothermally active contractile polymers;

FIGS. 15A-15B are charts showing the average temperature and temperature deviation, respectively, for the various hybrid electrothermally active contractile polymers;

FIG. 16A is a graph illustrating the correlation between temperature, length change and force of a hybrid electrothermally active contractile polymer;

FIG. 16B is a graph illustrating the stroke and force of various hybrid electrothermally active contractile polymers;

FIGS. 17A-17C are charts showing the work output, volumetric work output and the average work output for different hybrid electrothermally active contractile polymers;

FIG. 18A illustrates hybrid electrothermally active contractile polymers woven into an artificial muscle device;

FIGS. 18B-18C illustrate different weaving patterns of the hybrid electrothermally active contractile polymers used to make artificial muscle devices;

FIG. 19A illustrates an artificial muscle device active portions and passive portions,;

FIG. 19B illustrates a weaving pattern of hybrid electrothermally active contractile polymers and passive supporting fibers of an artificial muscle device;

FIG. 20 is a chart illustrating force and stroke distance versus time.

FIGS. 21A-21D are charts showing the force, strain, and temperature of coated and uncoated hybrid electrothermally active contractile polymers;

FIGS. 22A and 22B illustrate knitting of hybrid electrothermally active contractile polymers and non-conductive supporting fibers according to the present invention.

FIGS. 23A-23C and 23E-23F illustrate example devices of the present invention having electrothermally active contractile polymers having various configurations/orientations.

FIGS. 23D illustrates a device of the present invention having electrothermally active contractile polymers and one or more sensors.

FIGS. 23G-23H illustrate example devices of the present invention having electrothermally active contractile polymers arranged in a sack configuration.

FIGS. 24A-24E illustrate cross section example arrangements of artificial muscles comprising electrothermally active contractile polymers, the muscles being interconnected by one or more intermediary members comprising connectors, sensors, elastic members, lubricious members, insulating members and the like.

### DETAILED DESCRIPTION

The detailed description and specific examples contained herein, while indicating example embodiments of the apparatus, systems and methods, are intended only for the purpose of illustration and are not intended to limit the scope or breadth of the invention. Features, aspects, and advantages of the present invention are discussed in the following description, claims, and the accompanying figures. The figures are for illustration purposes only and are not drawn to scale. Identical reference numbers are used throughout the figures and description to indicate same or similar parts.

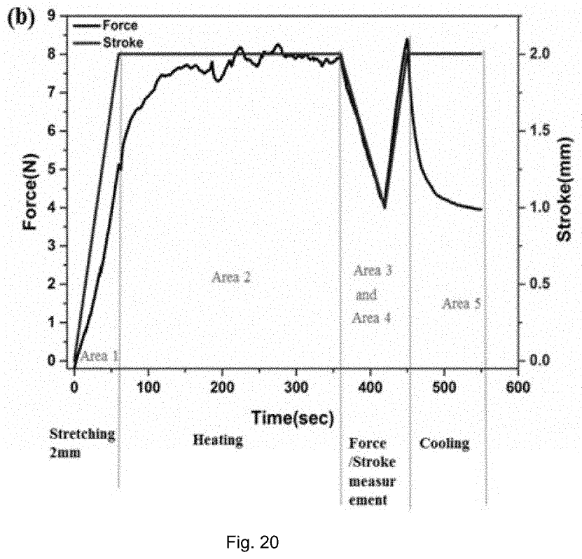

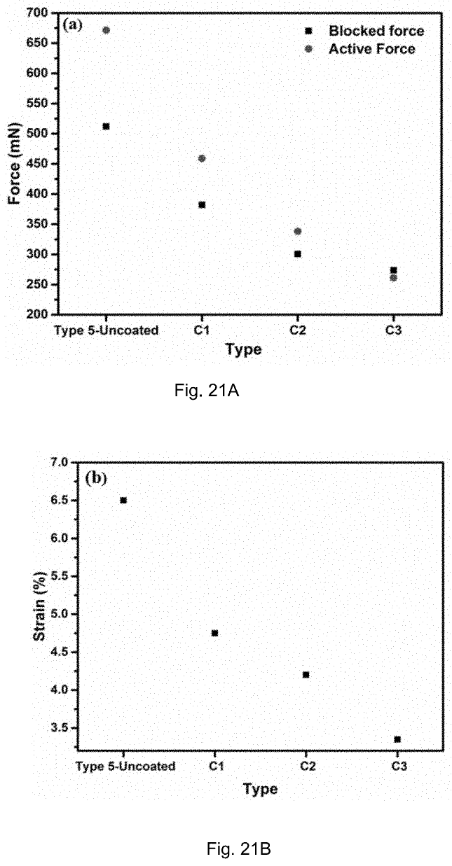

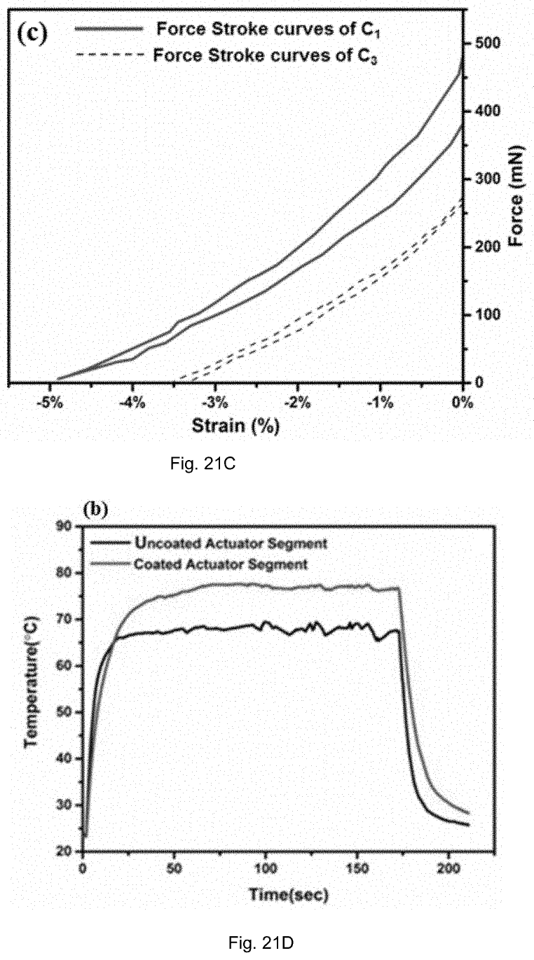

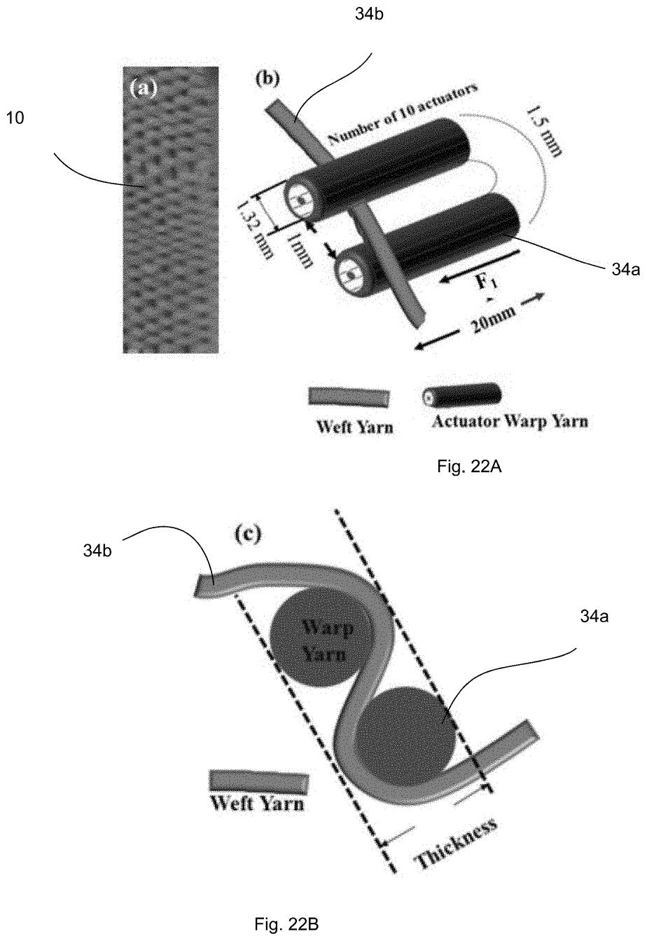

The present invention, illustrated in FIGS. 1-24E is directed to novel medical devices manufactured from artificial muscle fibers that comprise electrothermally active contractile polymers. The electrothermally active contractile polymers or actuators are made having various physical characteristics that, when used to produce an artificial muscle device of the present invention, can be implanted. The configuration of the electrothermally active contractile polymers allow for the tailoring or selective actuation of the devices of the present invention. The selective actuation includes but is not limited to variations in: 1) applied force, 2) direction of applied force, 3) work, 4) heat generation, 5) location of heat dissipation, and 6) length of actuation. The selective actuation enables a more precise control of a function of any anatomical structure such as the heart.

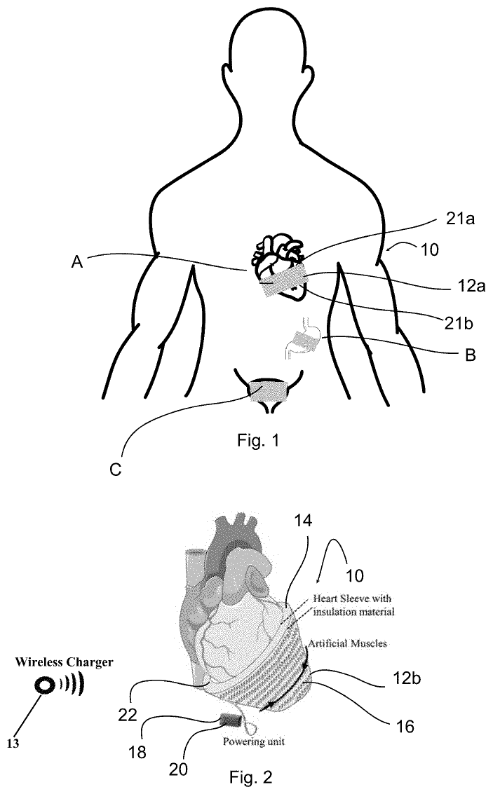

FIG. 1 illustrates an example embodiment of a new artificial muscle device 10 capable of more versatile and selective contraction. The new artificial muscle device 10 can be used to treat any number of conditions, such as heart disease (A), gastric disorders (B), and urinary tract disorders (C). In one example embodiment, as illustrated in FIG. 2, the artificial muscle device 10 includes a substrate 14 comprising one or more electrothermally active contractile polymers 16 that are connected together to form the artificial muscle device 10. The electrothermally active contractile polymers or fibers 16 are capable of imparting an action, such as a contracting force onto a tissue, organ, or bone to be treated.

The artificial muscle device 10 also includes a power supply 18 and a controller 20. In one example embodiment of the present invention, the power supply 18 and/or controller 20 are contained within a housing that can be implanted or secured external to a patient. The housing, while illustrated in FIG. 2 as being connected to the artificial muscle device 10 by a wire, may also use wireless technology to allow charging and control of the artificial muscle device 10. The controller 20 can be in wired and wireless communication with the artificial muscle device 10. The controller 20 can use any currently known or to be invented communication protocol including but not limited to Bluetooth®, WiFi, Z-Wave, Cellular (e.g., 3G, 4G, 5G, LTE), NFC, RFID, and the like. In one example embodiment of the present invention, the artificial muscle device 10 includes a remote or wireless charger 13 that is able to wirelessly charge the artificial muscle device 10. The wireless charger 13 can take any form including but not limited to a puck, mat, or a pad that can be placed in generally close proximity to the artificial muscle device 10. A belt or other securing device can be used to removably secure the wireless charger 13 in place against a patient's skin. The charging process can use any currently known or to be invented charging technology such as induction, radio, and resonance.

Referring to FIG. 1, the substrate 14 comprises any material that is capable of receiving and/or supporting the electrothermally active contractile polymers or fibers 16. A medical grade silicone material that is capable of being implanted can be used as the substrate 14. The substrate 14 can be manufactured in various shapes. The artificial muscle device 10, with or without substrate 14, can be configured as a sleeve 12a having generally open opposed ends 21a and 21b such that the artificial muscle device 10 can extend about an organ such as a heart, bladder, or bowl. The artificial muscle device 10 can also comprise a textile material that can be woven or knitted.

Referring to FIG. 2, the artificial muscle device 10, with or without substrate 14, can be configured as a pouch 12b having a single opening 22 toward an interior that is able to receive at least a portion of an organ such as a heart. As illustrated in FIGS. 1 and 2, the configurability of the artificial muscle device 10 lends itself to treatment of various organs. For instance, a sleeve 12a or pouch 12b may be used as a VCAD with the heart. A sleeve 12a may also be used to treat gastric or urinary tract disorders.

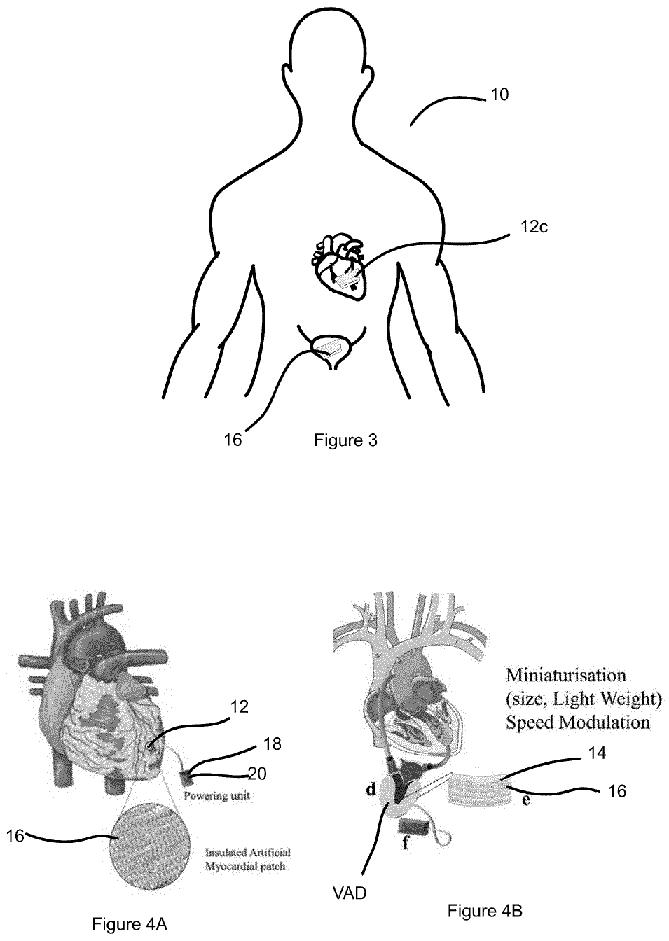

Referring to FIGS. 3 and 4A, the artificial muscle device 10 can be configured in the form of a patch 12c having electrothermally active contractile polymers or fibers 16 in operative communication with the power supply 18 and the controller 20. The patch 12c can be configured with or without substrate 14. The patch 12c is useful for organs and tissues with a specific location or disease. The patch 12c can be attached to tissue or a portion of an organ such as the bone, heart, stomach, or bladder in need of therapy. Any of the artificial muscle devices 10 can be secured in place by surgical suturing, staples, adhesives, and the like.

Referring to FIG. 4B, the artificial muscle device 10 can be in the form (generally identified by 12) of a sleeve 12a, pouch 12b, patch 12c or another configuration that is able to be coupled to or placed about an external or in vitro VAD. The artificial muscle device 10 and the electrothermally active contractile polymers 16 operate the VAD by compressing and decompressing a reservoir of blood to circulate it through the patient.

Turning now to the electrothermally active contractile polymers or fibers 16 that make up the artificial muscle device 10, each of the polymers or fibers 16 is able to move from a first position (e.g., non-contracted state) to a second position (e.g., contracted state), such as in a contraction or contractile movement. They can also move back from the second position to the first position to complete a full cycle. As illustrated in FIGS. 1 and 2, electrothermally active contractile polymers or fibers 16 can be arranged in various configurations. Additionally, they can be positioned on, under, or within the substrate 14. For instance, the electrothermally active contractile polymers or fibers 16 can be disposed circumferentially about a sleeve or pouch substrate 14. In this configuration, the electrothermally active contractile polymers or fibers 16 are able to contract and squeeze all or a portion of an organ such as the heart. In this way, the artificial muscle device 10 can act as a VACD and the electrothermally active contractile polymers or fibers 16 act as artificial muscle fibers.

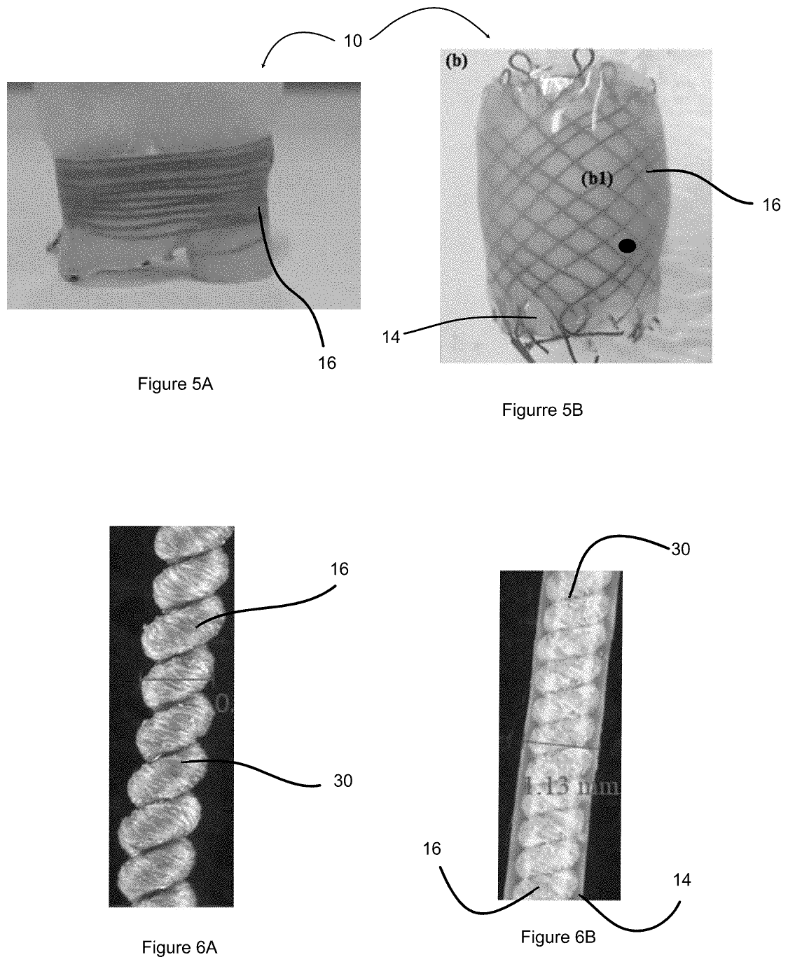

As mentioned above, the electrothermally active contractile polymers or fibers 16 can also be knitted into various configurations and patterns. For example, the electrothermally active contractile polymers or fibers 16 can be formed into a parallelly arrangement (FIG. 5A), or mesh sleeve (FIG. 5B). In a mesh configuration, the electrothermally active contractile polymers or fibers 16 are arranged in various angular orientations with respect to one another.

The contractile polymers or fibers 16 can also be used to fabricate device 10 with various fiber 16 architectures. For instance, electrothermally active contractile polymers or fibers 16 oriented in one direction may have a greater contractile force than electrothermally active contractile polymers or fibers 16 oriented in a different or angular direction. As a result, the artificial muscle device 10 of the present invention is able to provide more tailored or custom devices, implants, and treatments for personalized therapies. As will be discussed more fully below, other properties of the electrothermally active contractile polymers or fibers 16 can also be controlled, including but not limited to an amount or location of heat generation/dissipation and stroke (or actuation) length/distance.

As mentioned above, while a substrate 14 is not required it can provide some advantages in certain situations. When substrate 14 is used, the electrothermally active contractile polymers or fibers 16 can be positioned anywhere within, on, or on the substrate 14. For instance, as illustrated in FIG. 5A, and FIG. 5B the electrothermally active contractile polymers or fibers 16 can be disposed on an inner surface of substrate 14 such that they are generally positioned against or in close proximity to the tissue or organ being treated. Similarly, as illustrated in FIG. 2, the electrothermally active contractile polymers or fibers 16 can be disposed on or near an outer surface of substrate 14 such that they generally creating a space or insulating layer of substrate 14 between the electrothermally active contractile polymers or fibers 16 and the tissue or organs being treated.

The construction of the electrothermally active contractile polymers or fibers 16 (as described in more detail below provides a high degree of polymer/fiber alignment of synthetic fibers giving them a high amount of strength. Moreover, twisting of these polymers or fibers 16 to form a helical pattern causes an amplified thermally persuaded length change and corresponding force generation. The giant or increased force actuation of these electrothermally active contractile polymers or fibers 16 is achieved through a partial untwisting of the twisted polymers or fibers 16.

The electrothermally active contractile polymers or fibers 16 of the present invention are thermally driven making them candidates for many applications including, but not limited to biomedical devices and smart textiles. Their construction utilizes electrothermal energy as a clean source of energy for their operation. Additionally, the improved properties of the electrothermally active contractile polymers or fibers 16 and their ability to be electrothermally driven as active components make them an excellent artificial heart muscle (“AHM”) that may improve or replace the current VAD of the present invention.

Turning now to FIGS. 6A and 6B, the electrothermally active contractile polymers or fibers 16 can be fabricated or made of electrothermally driven synthetic fibers 16 coated with a conductive material such as silver. In one example embodiment, the synthetic fibers 16 comprise a silver coated nylon (SCN) fiber. The silver coating makes the synthetic fibers 16 highly conductive. Any conductive material may be used to coat the polymers or fibers 16.

Referring to FIG. 6B, the electrothermally active contractile polymers or fibers 16 can also be individually coated with an insulating material such as silicone. The silicone coated electrothermally active contractile polymers or fibers 16 provide electrical insulation between adjacent electrothermally active contractile polymers or fibers 16. Additionally, a silicone elastomer identical to or different from the silicone on the electrothermally active contractile polymers or fibers 16 can be used to interconnect the electrothermally active contractile polymers or fibers 16 in the formation of the artificial muscle device 10. While not required, ideally the interconnecting material will be a soft, stretchy, and flexible material akin to human flesh. This allows the electrothermally active contractile polymers or fibers 16 to move in a more natural way closer to the movement of human tissue and organs.

As mentioned above, the artificial muscle devices 10 of the present invention may include a VCAD placed around a diseased heart. The VCAD artificial muscle device 10 comprises the electrothermally active contractile polymers or fibers 16 extending about the heart. When activated, controller 20 causes voltage of approximately 13 V-32.5 V to be supplied from the power supply 18 to one or more of the electrothermally active contractile polymers or fibers 16. The electrothermally active contractile polymers or fibers 16 are then evenly heated to a temperature of approximately 48° C.-65° C. causing one or more of them to contract creating a pressure of approximately 120 mm Hg-140 mm Hg. The controller 20 then stops the supply of voltage, causing a decrease in temperature and a de-actuation of the electrothermally active contractile polymers or fibers 16. The controller 20 of the present invention is able to consistently operate the electrothermally active contractile polymers or fibers 16 of the artificial muscle device 10. While particular temperatures and voltages is discussed, other temperatures and voltages may be utilized to alter or change a characteristic of the device 10.

In another example embodiment of the present invention, a voltage of 2.1 V is applied to the electrothermally active contractile polymers or fibers 16 to produce 433 mN of force. Stress generated by individual electrothermally active contractile polymers or fibers 16 are generally 47 times that of natural cardiac filaments. It should be noted that the maximum stress of natural heart muscle is generated at the end of the systolic phase, which produces a blood pressure of approximately 120 mm Hg. The blocked stress of the electrothermally active contractile polymers or fibers 16 is approximately 89 kPa, which is fourfold as human heart muscle (“HHM”). As such, the electrothermally active contractile polymers or fibers 16 of the present invention have a higher performance compared to HHM. The stress generated by the electrothermally active contractile polymers or fibers 16 is approximately 89 kPa in the direction of the polymers 16 at 120 mm Hg, which is similar to the stress generated by natural heart muscle at end of systole phase.

In another example embodiment of the present invention, one or more sensors 17, such as a pressure sensor, can be integrated into the artificial muscle device 10. The sensor 17 can be in operative communication with the substrate 14, the electrothermally active contractile polymers or fibers 16, or a portion of organ being treated.

The sensor(s) 17 can also be configured to automatically control the electrothermally active contractile polymers or fibers 16 of the artificial muscle device 10. When power is applied to the electrothermally active contractile polymers or fibers 16, the pressure increases reaching an upper pressure limit of approximately 120 mm Hg, which is sensed by the sensor 17. As described above, when the sensor 17 detects the 120 mm Hg, it is able to communicate with the controller 20, which terminates the power causing deactivation of the electrothermally active contractile polymers or fibers 16. As the polymers or fibers 16 begin to cool a corresponding decrease in pressure begins and continues to a lower pressure limit of approximately 80 mm Hg. The sensor 17 is activated upon reaching the preset lower limit causing the electric power to be switched on again by the controller 20, thereby beginning another full cardiac cycle.

The electrothermally active contractile polymers 1 or fibers 6 are able to operate with an approximate frequency of 1.4 Hz, which is approximately 14 beats within 10 s, which is approximately 84 cycles per minute and can be altered as needed compared to 72 human heartbeat cycles. The ability of the artificial muscle device 10 of the present invention to control and vary heartbeat cycles provides cardiologist with the ability to tailor the device 10 to the particular needs of patients.

One example embodiment of the present invention includes artificial muscle device 10 having a size or dimension of approximately 1.13 cm×10 cm×0.2 cm and weighing only 3.9 g with a density of 1.73 g cm⁻³. The artificial muscle device 10 of the present invention is able to reduce the total weight and volume of artificial heart devices while maintaining pulsatility on LV assist devices.

Manufacturing of the Conductive Actuators

We will now turn to the fabrication and material of the individual electrothermally active contractile polymers or actuators 16 of the present invention. The electrothermally active contractile polymers or actuators 16 of the present invention are improved electroactive polymers having mechanical properties, durability, and abrasion resistance necessary for implantation and for generating a force necessary to control a diseased organ.

In an example embodiment, the electrothermally active contractile polymers 16 are manufactured from one or more fibers 30 that are combined and then coiled along a long axis of the fibers 30. The fibers 30 can comprise a polyester material that is capable of being readily converted to nonwoven textiles by various methods including by a wet-laid and melt-blown web forming process bonded with mechanical methods such as needle punching, chemical or thermal bonding. Nonwoven polyester textiles can be advantageously used in the present invention as they are strong, permeable, and resistant to stretching, heating, shrinking, abrasion, mildew and most chemicals. In one example embodiment, the fibers 30 have a linear density of 180 g/m2. However, the density may vary depending upon the desired active medical device 10 and its particular function.

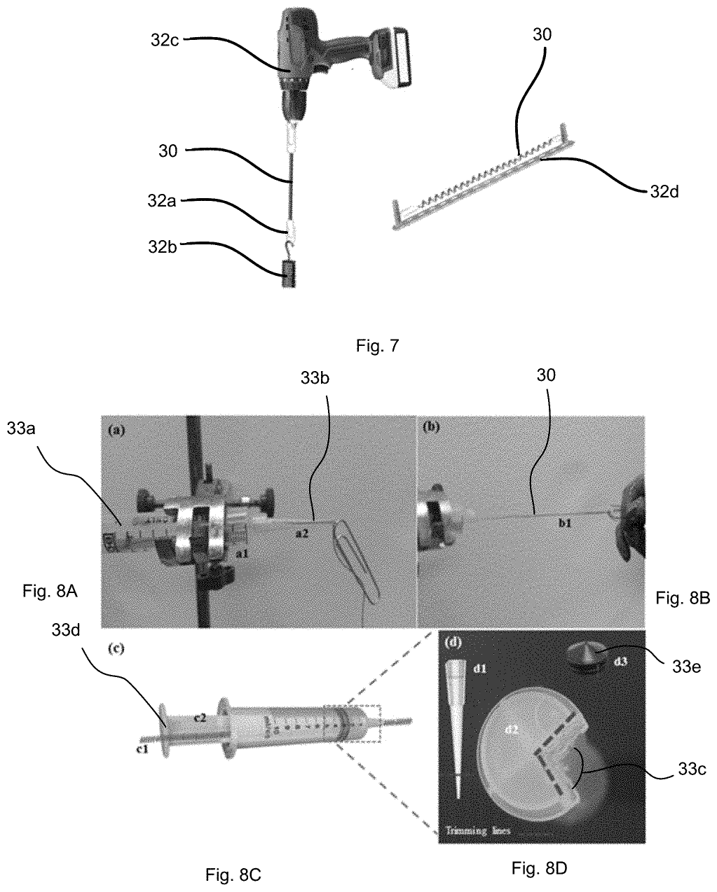

Turning to FIG. 7, an example of the coil fabrication process is illustrated that involves twisting the combined fibers 30 until it becomes coiled. As a first step, a length of precursor fibers 30 are cut off from a yarn spool and the ends of the fibers 30 are tied to a connector 32a that is coupled to and extends between the ends of the fibers 30 and an anti-rotation member 32b that prevents the ends of the fibers 30 connected to the connector 32a from rotating. In one example embodiment, the connector 32a may comprise a metallic paper clip, a hook, a clamp, and the like. In one example embodiment, the anti-rotation member 32b may comprise a weight having a hook member, where the weight is sufficient to prevent rotation of the connector 32a. The anti-rotation member 32b or weight is applied so that the fibers 30 are held in tension while also preventing their rotation. In another example embodiment of the present invention, the ends of the fibers 30 may be connected to a stationary member such as a wall, a post, or other non-movable or non-rotatable object.

The other end of the fibers 30 are then attached to a twisting device or mechanism 32c such as a power drill or other rotation imparting mechanism or device. The precursor or uncoiled fibers 30 are twisted by rotating the twisting device 32c or powered drill in either a clockwise direction (from the top view) to form a “S” twist or an anti-clockwise direction to form a “Z” twist.

The coiled fibers 30 can then be mounted on a stand 32d that is operated to stretch the coiled fibers 30 for approximately 8% with respect to the initial coiled length of the fiber 30. Lastly, the stretched coiled fibers 30 on the stand 32d are then thermally annealed at 200° C. for 1 hour to stabilize the actuator (stretched coiled fibers 30), which may then undergo a coating process. A coiling method was described in Haines, C. S., et al., Artificial Muscles from Fishing Line and Sewing Thread. Science, 2014. 343(6173): p. 868-872).

Once the fibers are coiled, coated, and annealed, they now act as the individual electroactive polymers or actuators 16 capable of exerting force upon a treated organ.

Electrical Insulation Coating for Conductive Actuators

In conventional actuating devices, electrical shorting of the actuating device will change the conductive pathways, which can result in inconsistent functionalities. As a precaution for electrical shorting, the present invention coats the fibers 30 with a coating such as silicone. While a coating material of silicone is discussed, it is also feasible to use other coating materials that prevent electrical shorting.

After the twisting and thermal annealing processes (discussed above), the actuator 16 is coated with a silicone material. The coating process, illustrated in FIGS. 8A-8D, can utilize the stand 32d used during the thermal annealing process. To apply the silicone coating a delivery device 33a such as a syringe, is filled with a soft silicone material (such as one provided by Smooth-On Inc Pvt. Ltd).

A control tip 33b, such as a pipette tip, is then connected to the delivery device or syringe 33a to direct a flow of the silicone material onto the actuator 16.

In another example embodiment of the invention, the actuator 16 is placed inside of a delivery device or syringe 33a having a control 33b. An end of the actuator 16 is passed through the control tip 33b and is then slowly pulled in a straight position, as pictured in FIG. 8B, out from the syringe 33a and control tip 33b. The pulling of the actuator 30 results in applying a silicone coating around the actuator 16.

In an example embodiment, the conductive actuator 16 was passed through a modified syringe 33a in-between a gap 33c formed in a plunger 33d and a syringe surface. A part of the syringe plunger 33d was removed, as shown in the cross section shown in FIG. 8(d) and a slit 33e was formed or inserted in rubber plunger top 33f to make a gap to pass the actuator 16 through the silicone material contained within the syringe 33a.

The coating thickness may be controlled by precision measuring and trimming of the pipette tip as shown in FIG. 8(d). Then the silicone coated actuator 30 is kept at room temperature for 1 hour to allow for curing. The actuator 16 is then annealed for a second time with 50% stretch. The coating thickness is controlled by maintaining various trimming position of the pipette tip 33b.

As discussed above, the conductive actuator 16 is prepared only with a first stage of thermal annealing by stretching it to around 8%. The second stage of annealing is completed after the coating as the 50% stretch during second annealing produced a gap in between adjacent turns in the coils. Applying the silicone substrate after this second stage of annealing would mean that the silicone material would fill the gaps between turns in the coil and restrict the actuator 16 movement and further diminish the actuator 16 performance.

Any number of coatings, application methods, and/or coating locations may be applied to the actuators 16. A different number of coatings, application methods, and/or coating locations generally impart a different characteristic to the actuator 16.

Changing Actuator Properties

Depending upon the functional needs of the artificial muscle device 10, a surface resistivity of the fibers 30 can be altered during the manufacturing process. In particular, the surface resistivity can be altered by controlling the number of coatings.

Hybrid Electrothermally Active Contractile Polymers

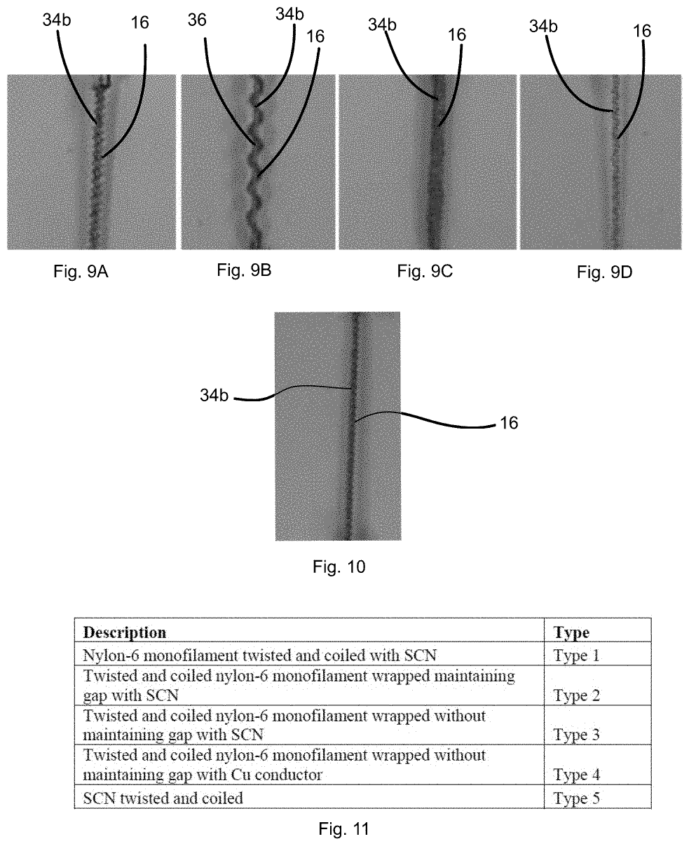

Turning to FIGS. 9A-9D, the electrothermally active contractile polymers or actuators 16 of the present invention also include sole or hybrid electrothermally active contractile polymers or actuators 16 having at least two fiber components 34a and 34b. One fiber component of the hybrid electrothermally active contractile polymers or actuators 16 is used for the purpose of actuation and is ideally a non-conductive fiber 34a, and the other fiber component is an electrothermal heating fiber 34b. The non-conductive fiber 34a and the electrothermal heating fiber 34b are twisted or coiled together to form the hybrid electrothermally active contractile polymers or actuators 16. In one example embodiment, monofilament nylon-6 may be used as the non-conductive fiber 34a while the electrothermal heating fiber 34b may comprise SCN yarn or graphene coated fibers 30.

In an example manufacturing process, a hybrid electrothermally active contractile polymers or actuators 16 of the present invention are fabricated with non-conductive fiber (e.g., monofilament nylon-6) 34a having a diameter of approximately 0.4 mm. The filament is co-twisted with the thermally conductive fiber (e.g., SCN yarn) 34b. The hybrid electrothermally active contractile polymers or actuators 16 are fabricated with 10 MPa stress calculated with respect to the sum of the cross-sectional areas of the fibers 30.

Referring to FIGS. 9A and 12A, the fibers 34a and 34b are twisted together to form an electrically conductive “Type 1” hybrid electrothermally active contractile polymer or actuator 16. This forms a non-identical plied yarn hybrid electrothermally active contractile polymer or actuator 16. FIGS. 9B, 12B and 9C and 13A, illustrate what's termed “Type 2” and “Type 3” hybrid electrothermally active contractile polymers or actuators 16. Each are fabricated by wrapping the electrothermal heating fibers (SCN) 34b around an already twisted and coiled non-conductive (nylon-6 monofilament) fiber 34a (shown in FIG. 12B). The conductive electrothermal heating fibers (SCN) 34b can be wrapped with and without a gap 36 (see FIGS. 9B and 12B) between the coils of the conductive electrothermal heating fibers (SCN) 34b. Different densities and configurations of gaps 36 are possible and should be considered to be within the spirit and scope of the present invention. Referring now to FIGS. 9D and 13B, a “Type 4”, hybrid electrothermally active contractile polymer or actuator 16 is fabricated with the same twisted and coiled monofilament fiber 34a but is also wrapped with a metal fiber or wire 38 such as copper (Cu). The wire 38 is a conductive element conserving gaps 36 between conductors.

Sole Hybrid Conductive Actuator

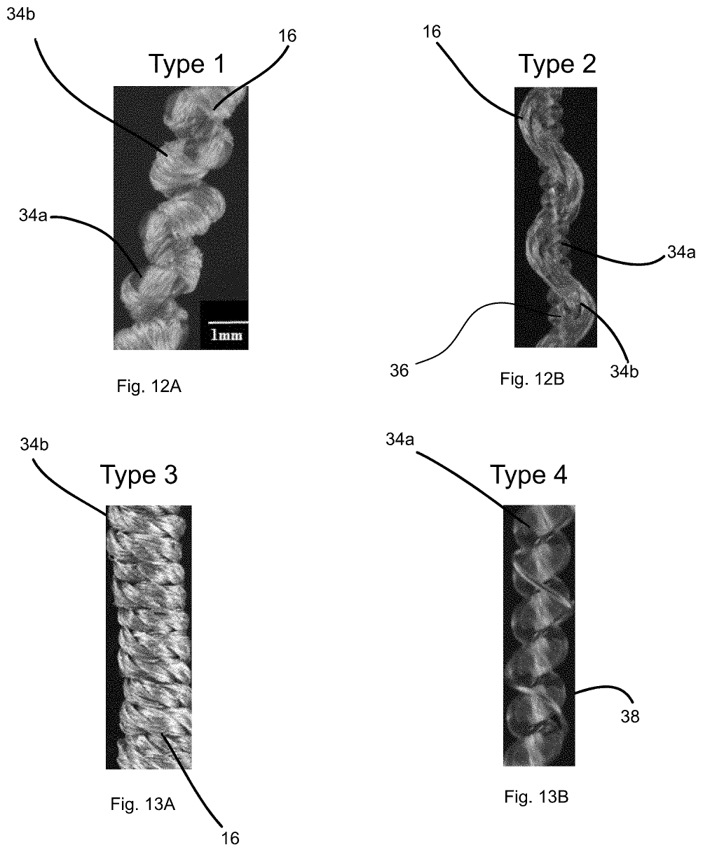

Turning now to FIG. 10 and FIG. 11, a sole hybrid electrothermally active contractile polymer or actuator 16 can also be used in the manufacture of the artificial muscle device 10. The sole hybrid electrothermally active contractile polymer or actuator 16 comprises electrically conductive fibers (SCN) 34b and non-coated fibers or yarns. The “Type 5” polymer 16 is fabricated by twisting the electrically conductive synthetic fibers (SCN) 34b under a 6 MPa stress, which is the minimal stress for the coiling polymer fabrication. The sole hybrid electrothermally active contractile polymer 16 allows for reduced manufacturing complexity and may be beneficial for patients with allergies to a material used in any of the hybrid polymers or actuators 16. A list of the different types of hybrid polymers or actuators 16 of the present invention are provided in table of FIG. 11.

Heating

The electrothermally active contractile polymers or actuators 16 of the present invention are able to generate an average temperature of 69.5° C. As described above, the polymers or actuators 16 are manufactured using both one or more polymer fibers 34a and conductive yarns 34b. The manufacturing methods of the present invention result in hybrid polymers or actuators 16 that provide even heating across or along its length. It should be noted that manufacturing hybrid electrothermally active contractile polymers or actuators 16 with a metal material such as steel or copper 38 may provide an increase in strength but decrease the even heating along the length of hybrid polymer or actuator 16.

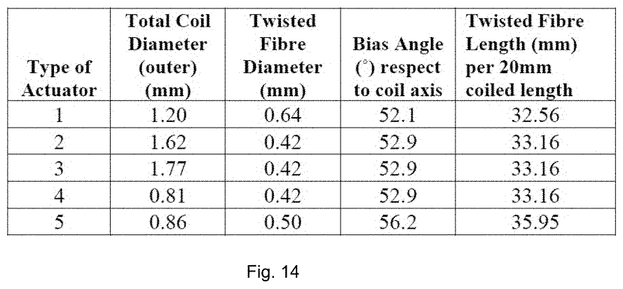

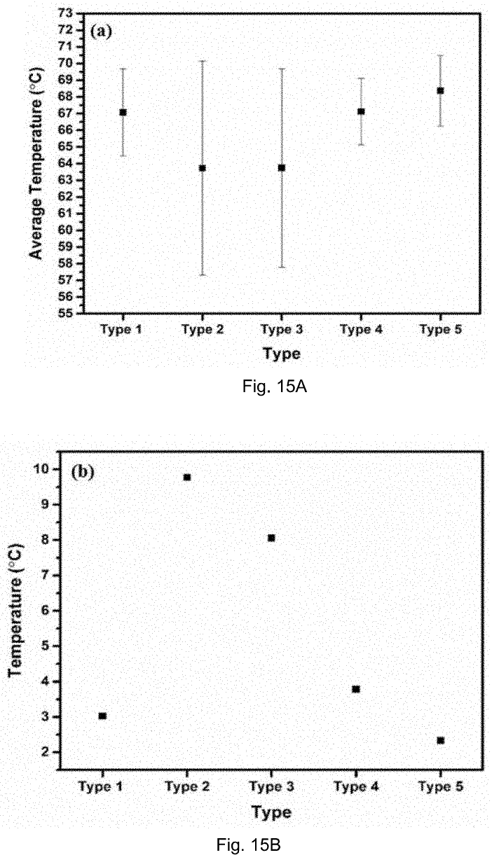

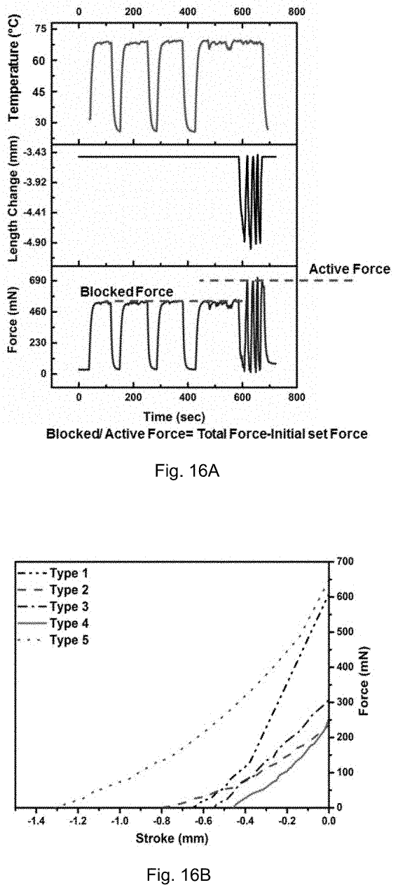

Turning to FIGS. 14, 15A, and 15B, it can be seen that the various hybrid electrothermally active contractile polymers or actuators 16 of the present invention provide a variety of physical and temperature producing characteristics that lends them to particular applications. For instance, the Type 5 sole hybrid electrothermally active contractile polymers or actuators 16 can be heated with a minimum variation of the temperature along its length. Further twisting of the SCN yarn 34b in the same direction of the twisting of the raw or un-coated/fibers yarn improves the conductive pathways and conductivity which provides an even temperature distribution. The stability of the Type 5 sole hybrid electrothermally active contractile polymers or actuators 16 makes it ideal in temperature sensitive environments such as the improved VCAD artificial muscle device 10 of the present invention.

The Type 2 and Type 3 hybrid electrothermally active contractile polymers or actuators 16, as shown in FIGS. 15A and 15B, have the largest temperature variance along their length, which is due to the nonwoven textiles or fibers 30. The Type 2 and 3 hybrid polymers 16 are ideally used where variation in temperature is not a concern. These hybrid polymers or actuators 16 are more cost effective and generally easier to manufacture.

Force and Work of Hybrid Conductive Actuators

Turning to FIG. 16A, the novel hybrid electrothermally active contractile polymers or actuators 16 of the present invention are able to generate a force of approximately 690 mN. The force is calculated by deducting an initial set force which of approximately 33 mN from a total force of the polymers or actuators or actuators 16. A stroke value (AL) is able to be calculated with respect to the initial set position and strain %. The stroke value is calculated by normalizing the stroke values to the initial unloaded length (e.g., 20 mm) of the actuator. The force and strain data are important demonstrating the benefits and performance of the hybrid electrothermally active contractile polymers or actuators 16 and the devices 10 to which they are incorporated.

FIG. 16B illustrates the stroke and force of the various hybrid electrothermally active contractile polymers or actuators 16 of the present invention. It should be noted that the Type 5 sole hybrid polymer/actuator or actuators 16, which as described above has the most stable temperature characteristic, also has the highest force with approximately 690mN. The stable temperature and increased force properties of the Type 5 sole hybrid polymer/actuator 16 makes it ideal for implantable medical device products.

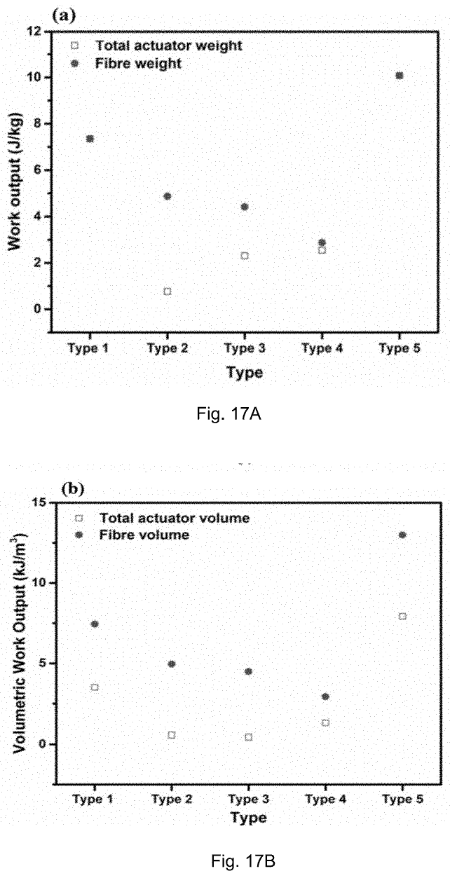

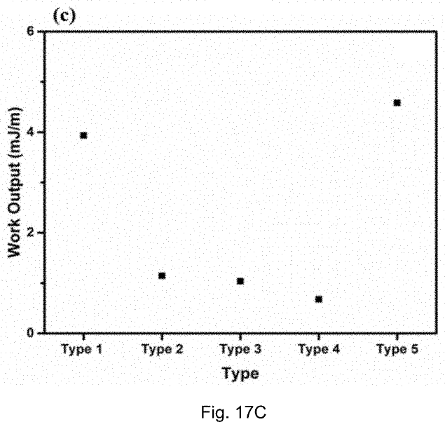

FIGS. 17A-17C illustrate the various work per unit weight, unit volume with reference to the twisted fiber and actuator, and unit length with reference to the hybrid conductive actuators 16 length (approximately 20 mm). FIGS. 17A and 17B, demonstrate that the gravimetric and volumetric work output of the hybrid electrothermally active contractile polymers or actuators 16, with identical actuating material and different heating techniques, exhibit a maximum difference of approximately 41% between “Type 2” and “Type 4” hybrid polymers or actuators 16 normalized to the weight and volume of twisted fiber 30. FIG. 17C demonstrates the work output per length also varies among the different hybrid polymers or actuators 16 of the present invention. As can be seen, the maximum difference of the work output per length between three hybrid polymers or actuators 16 types was 41% between “Type 2” and “Type 4” actuators.

An important aspect of the present invention is that the type of construction of the hybrid electrothermally active contractile polymers or actuators 16, allow for selection of hybrid polymer/actuator 16 characteristics. For instance, having an electrothermally heated fiber (such as SCN) 34b, with and out gaps 36, wrapped about a coiled conductive actuator fiber (such as nylon-6 monofilament) 34a, can be heated within the same range of temperature, and exhibit the same work outputs, by provide different force and stroke values (see FIG. 16B). Therefore, different hybrid electrothermally active contractile polymers or actuators 16 can be used to knit the artificial muscle device 10 to impart varying device properties. The “Type 5”, sole hybrid electrothermally active contractile polymers or actuators 16, which as described above is fabricated with only SCN yarn 34b, provides the lowest stiffness and the highest stroke. Further, the “Type 5” sole hybrid polymers or actuators 16 provides approximately 2-fold larger actuation stroke than the “Type 4” and “Type 1” actuators 16. As discussed above, the “Type 5” sole hybrid polymers or actuators 16 provides the highest performance parameters including gravimetric, volumetric, per length work output, and stroke values.

Insulation

To prevent shorting of the hybrid electrothermally active contractile polymers 16 of the present invention, some or all of the hybrid polymers/actuators 16 are coated or insulated with a non-electroactive material 14 such as silicone. Ideally, a soft silicone (such as Smooth-On Inc Pvt. Ltd) is used. The insulating coating process begins by slowly pulling one or more hybrid electrothermally active contractile polymers or actuators16 in a generally straight or uncoiled position. This results in applying an insulating coating around the entire surface area of the hybrid electrothermally active contractile polymers or actuators 16. The silicone coated polymers/actuators 16 are then allowed to cure for approximately 1 hr. at room temperature. Other insulating materials and drying processes are also contemplated herein and should be considered to be within the spirit and the scope of the present invention.

It should be noted that the thickness of the insulating coating may be modified in order to alter one or more properties of the hybrid electrothermally active contractile polymers or actuators 16. For instance, a thicker coating of insulating material 14 can be applied if all or part of the hybrid electrothermally active contractile polymers or actuators 16 generate heat higher than desired in a particular application. In another instance, a thicker coating of insulating material 14 may be applied to limit a stroke length or force of a hybrid electrothermally active contractile polymers or actuators 16 requiring an increased voltage for operation.

In another example embodiment of the present invention, hybrid electrothermally active contractile polymers or actuators 16 can have uncoated and coated actuator segments that are heated with the same voltage. The coated actuator segments demonstrate a lower temperature than the un-coated actuator segments as the silicone coating acts as a thermal insulation. Due to the low thermal conductivity of the coating or insulation 14, such as silicone, heat generated by the hybrid electrothermally active contractile polymers or actuators 16 is not transmitted to the surface of the coating and likewise to the surface of an organ or a patient's skin.

An advantage of having coated and un-coated actuator segments is that the artificial muscle device 10, can be manufactured with un-coated actuator segments in non-critical locations, such as non-tissue contacting areas. Heat generated by the hybrid electrothermally active contractile polymers or actuators 16 are able to escape through the un-coated actuator segments, thereby assist to dissipate heat.

Knitting of Implantable Devices

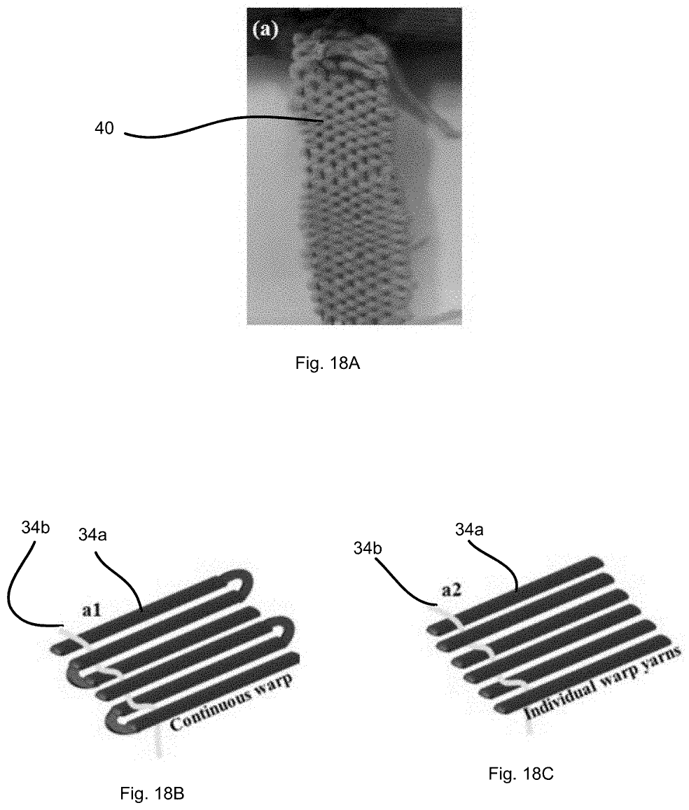

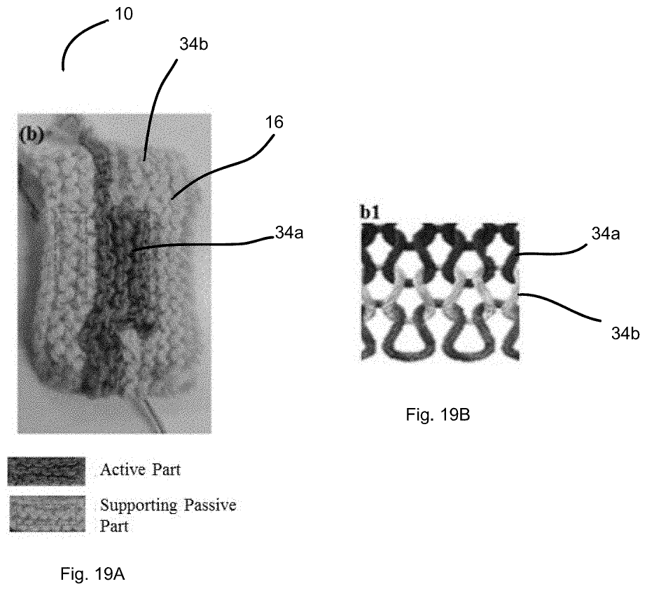

Referring to FIGS. 18A-19B, 22A and 22B, the present invention includes hybrid electrothermally active contractile polymers or actuators 16 that are flexible and can be manufactured into knitted active medical devices 40, including those that can be implanted. The knitting or weaving process includes continuous warp yarn knitting and individual yarn knitting as illustrated in FIGS. 18B and 18C. The woven fabrics of the present invention are created on a loom by interlacing threads forming right angles to one another. Weaving of hybrid electrothermally active contractile polymers or actuators 16 is done by either of the two methods. While the figures illustrate specific yarns/fibers as conductive fibers 34a and electrothermally conductive fibers 34b, it should be understood that they are interchangeable to provide the artificial muscle device 10 with different characteristics. An example woven textile is prepared with 20 mm×23 mm as the dimensions of length (Ltex)×width (wtex). As can be seen in FIG. 22B the width of the textile of the artificial muscle device 10 is equal to the sum of the diameters of the hybrid conductive actuators 16 (1.32 mm) and the gap in between the hybrid electrothermally active contractile polymers 16 is 1 mm. The total width (wtex) is approximately 23 mm as illustrated in the below equation.

wtex=(1.32*10+(1*(9))=23 mm

The thickness of the woven textile used to manufacture the artificial muscle device 10 of the present invention is the thickness of the hybrid conductive electrothermally active contractile polymers or actuators 16 (warp yarn) and two times of the thickness of the weft cotton yarn as can be seen in the cross section of the woven textile in FIG. 22B. The total thickness (t) of the woven textile is 3.32 mm as given by the following equation. The total length of the actuator 16 used for the textile fabrication was around 215 mm.

t=(1.32+(1*2))=3.32 mm

The force generated by one actuator is F₁ and the number of parallel actuators in a textile is n_{w}, the force generated by the woven textile (F_{w}) is equal to the sum of parallel forces given by below equation

F_{W}=F₁*n_{w}

The strain of the single actuator (ΔL %) and the strain of the woven textile is (ΔL_{w} %) are equal.

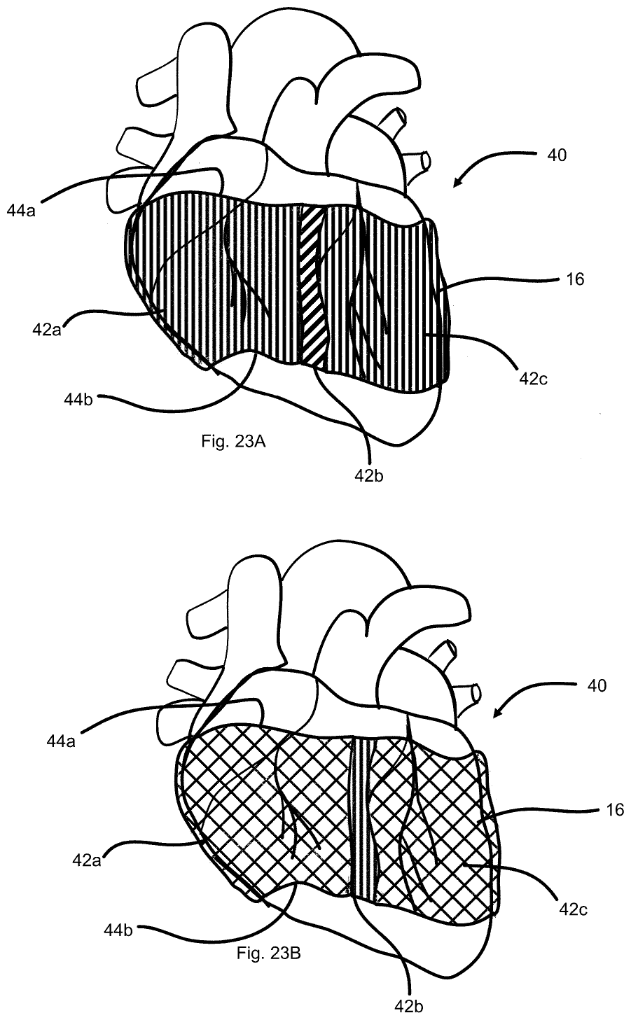

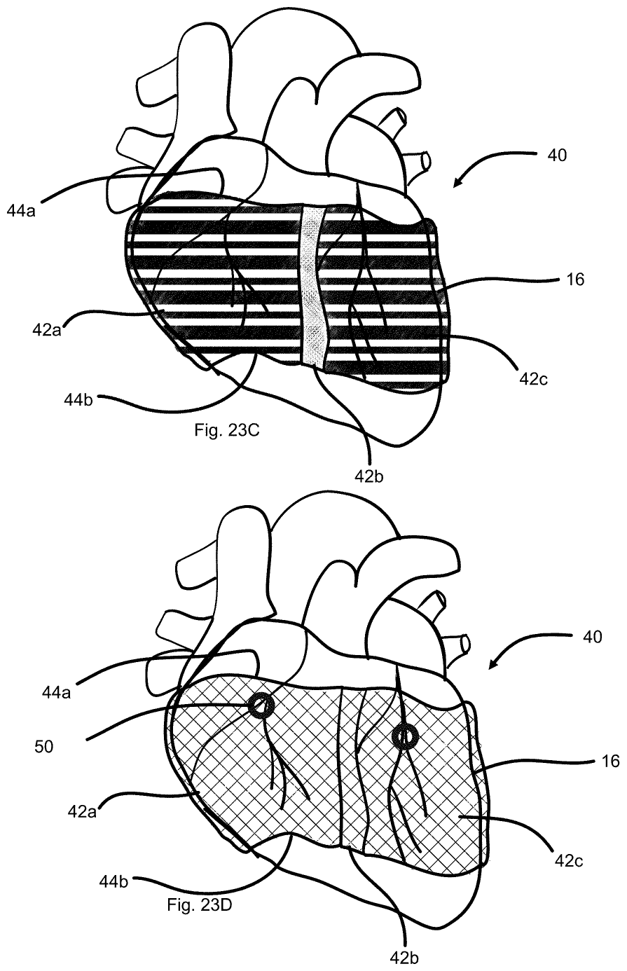

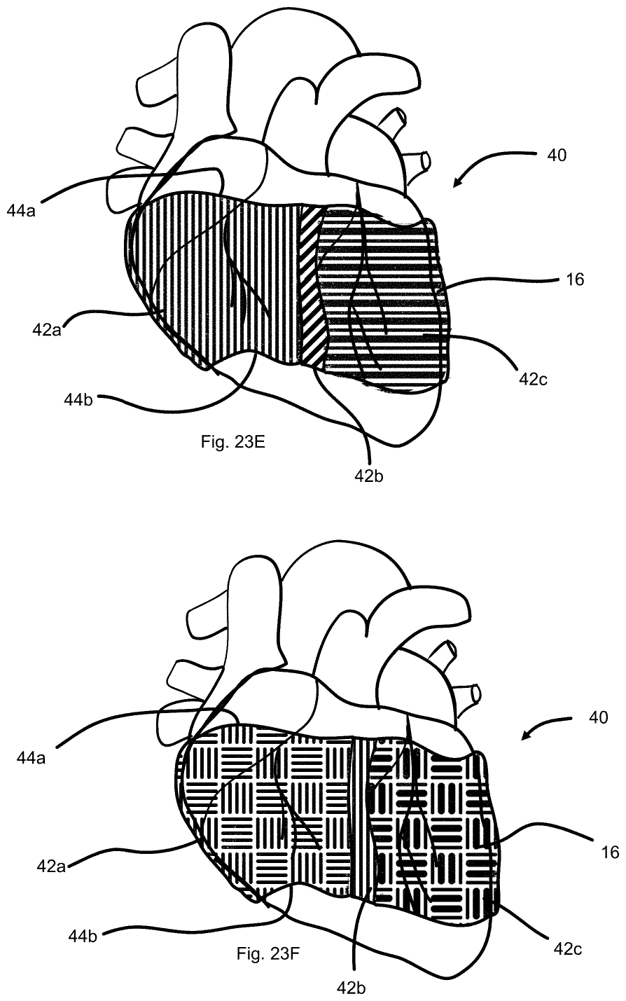

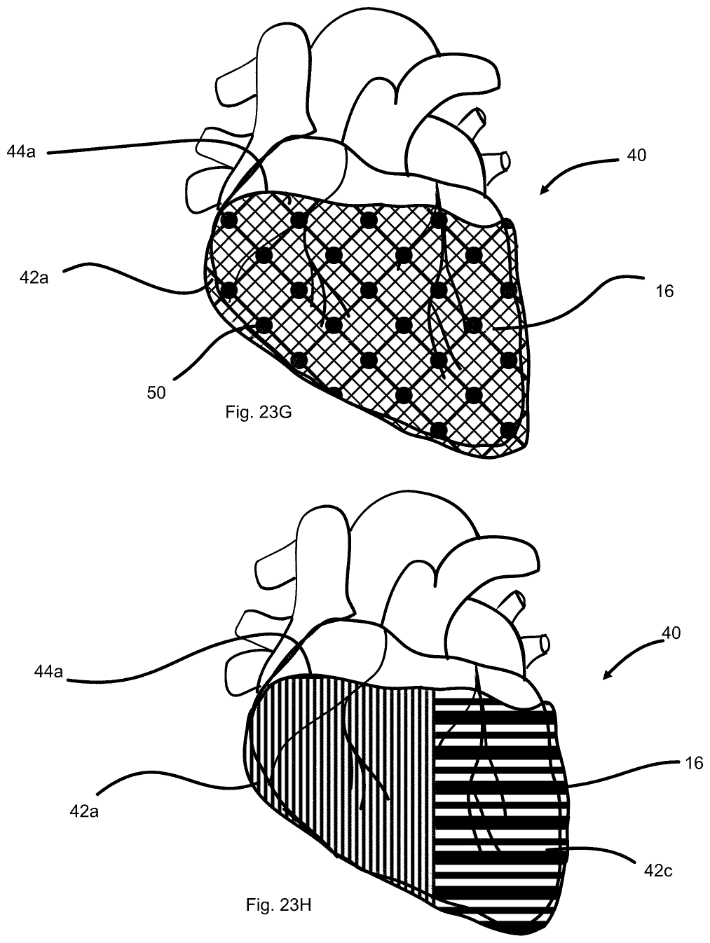

FIGS. 23A-23C and 23E-23F illustrate example medical devices 40 of the present invention having electrothermally active contractile polymers or actuators 16 having various configurations/orientations. FIG. 23A for example, illustrates a medical device 40 that extends about an organ such as the heart. The medical device 40 is divided into at least 3 sections, including a left section 42a, a middle section 42b, and a right section 42c. The left section 42a and the right section 42c of the medical device 10 is comprised of electrothermally active contractile polymers or actuators 16 in a generally vertical arrangement while the middle section 42b is comprised of electrothermally active contractile polymers or actuators 16 or non-electrothermally active contractile polymers or actuators 16 in a generally non-vertical arrangement.

The medical device 40 may include one or more selvages or ribs 44a and 44b that are spaced apart and to which one or more of the electrothermally active contractile polymers or actuators 16 connect or attach. One or more interconnecting ribs 44c may extend between the spaced apart ribs 42a and 42b to aid in reducing or preventing movement away from each other. One or more of the selvages or ribs 42a-42c may be electrothermally active contractile polymers or actuators 16 or non-electrothermally active contractile polymers. Additionally, any number of selvages or ribs may be used in the construction of the medical device 40.

Referring to FIG. 23B, the example medical device 40 of the present invention is comprised of a left section 42a and a right section 42c having electrothermally active contractile polymers or actuators 16 arranged in a mesh configuration. The middle section 42b is comprised of electrothermally active contractile polymers or actuators 16 or non-electrothermally active contractile polymers in a generally vertical arrangement.

Referring to FIG. 23C, the example medical device 40 of the present invention is comprised of a left section 42a and a right section 42c having electrothermally active contractile polymers or actuators 16 arranged in a generally spaced apart horizontal configuration. The middle section 42b is comprised of electrothermally active contractile polymers or actuators 16 or non-electrothermally active contractile polymers in a generally mesh arrangement.

Referring to FIG. 23D, the example medical device 40 of the present invention is comprised of a left section 42a and a right section 42c having electrothermally active contractile polymers or actuators 16 arranged in a mesh configuration. The middle section 42b is also comprised of electrothermally active contractile polymers or actuators 16 or non-electrothermally active contractile polymers in a mesh arrangement or configuration.

The medical device 40 of FIG. 23D also includes one or more sensors 50 that are capable of measuring a number of physiological parameters, including but not limited to cardiac pressures such systolic pressure, diastolic pressure, and afterload. The sensors 50 can comprise one or more nanotubes that incorporated into the medical device 40 and adapted to send/receive signals with the controller 20, which can be transmitted for monitoring.

Referring to FIG. 23E the example medical device 40 of the present invention is comprised of a left section 42a having electrothermally active contractile polymers or actuators 16 arranged in a vertical arrangement and a right section 42c comprised of electrothermally active contractile polymers or actuators 16 in a generally horizontal arrangement. The middle section 42b is comprised of electrothermally active contractile polymers or actuators 16 or non-electrothermally active contractile polymers in a generally non-vertical arrangement, but any configuration may be used.

Referring to FIG. 23F the example medical device 40 of the present invention is comprised of a left section 42a and a right section 42c having electrothermally active contractile polymers or actuators 16 arranged both vertical and horizontal arrangements and a middle section 42b comprised of electrothermally active contractile polymers or actuators 16 or non-electrothermally active contractile polymers in a generally vertical arrangement. The electrothermally active contractile polymers or actuators 16 of the right section 23c are generally thicker providing a different functional property than the left section 42a. For instance, the electrothermally active contractile polymers or actuators 16 may have increased force and/or less travel. Other functional properties are also possible. FIGS. 23G and 23H illustrate sock, sack, or pouch configurations of the medical devices 40 of the present invention. In these embodiments, a single opening 22 is provided for accessing an interior of the medical device 40. A rib 44a may extend about the opening 22 defining the access opening. A bottom of the medical device 40 is entirely or partially covered by electrothermally active contractile polymers or actuators 16 or non-electrothermally active contractile polymers. One or more sensors 50 may also be incorporated into the medical device 50 to monitor one or more physiological or device 40 parameters.

As particularly illustrated in FIG. 23H, there is no requirement for the medical device 40 to have one or more sections. There is also no requirement for a middle section 42b, as illustrated in FIG. 23G and FIG. 23H. While example left, right, and middle sections have been disclosed herein, it should be appreciated that the medical devices 40 of the present invention can comprise one or more sections positioned or arranged in any direction, location, configuration, or arrangement.

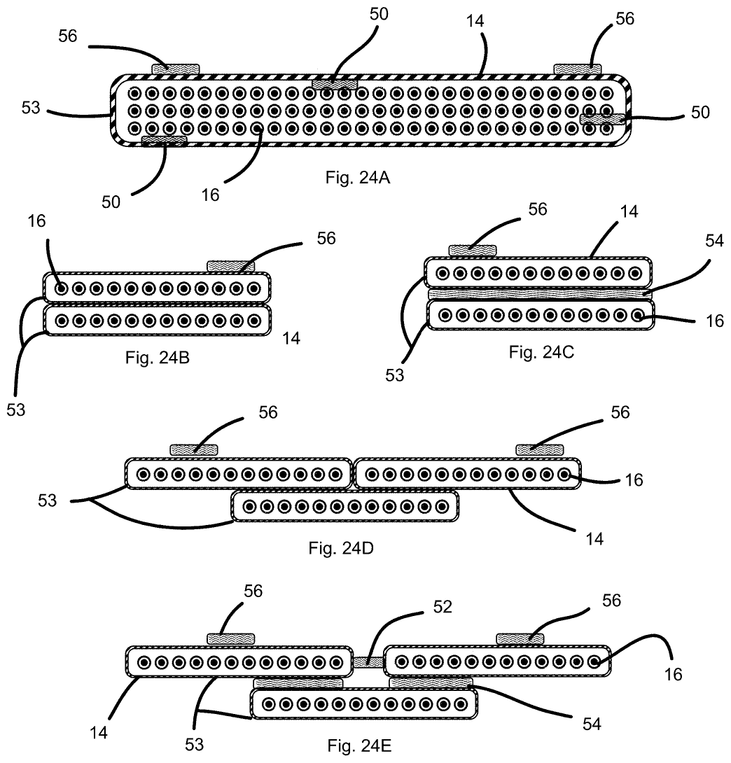

Turning to FIGS. 24A-24E, cross section example arrangements of artificial muscles according to the present invention are illustrated. The artificial muscles can comprise one or more layers of electrothermally active contractile polymers or actuators 16 that may or may not be individually coated or collectively coated. The electrothermally active contractile polymers or actuators 16 of the artificial muscles can be imbedded in a substrate 14 to protect or isolate the tissue ingrowth and/or to protect surrounding tissue from heat and movement of the device 40.

One or more sensors 50 can be imbedded or attached to the substrate 14 or electrothermally active contractile polymers 16. Placement of one or more sensors 50 can measure or detect various physiological and/or device parameters.

As illustrated in FIGS. 24C and 24E, the artificial muscles can be separated or connected by one or more intermediary members 52 that can comprise connectors, sensors, elastic members, lubricious members, insulating members and the like. The intermediary members 52 can function to allow different artificial muscles to move respect to each other. In some example embodiments, the intermediary members 52 may comprise electrothermally active contractile polymers or actuators 16 to allow active control of movement between the artificial muscles.

The present invention may be embodied in other specific forms without departing from the spirit or essential attributes thereof, and it is, therefore, desired that the present embodiment be considered in all respects as illustrative and not restrictive. Similarly, the above-described methods and techniques for forming the present invention are illustrative processes and are not intended to limit the methods of manufacturing/forming the present invention to those specifically defined herein.

What is claimed is:

1. An artificial muscle construct positionable in contact with an organ requiring therapy, the construction comprising:
thermally active actuators interconnected to form an implantable medical device, each of the thermally active actuator being configured to move axially between a contracted state and an extended state;
an insulating layer extending about at least a portion of the thermally active actuators to protect at least a portion of the organ from heat;
a controller in communication with the thermally active actuators to control their movement between the contracted state and the extended state;
a power supply configured to supply power to the controller and the thermally active actuators; and
wherein at least a portion of the thermally active actuator being configured to impart or remove a force onto the organ.

2. The artificial muscle construct device of claim 1, wherein each of the thermally active actuators comprise a conductive coiled fiber configured to move from the extended state to the contracted state when powered.

3. The artificial muscle construct device of claim 1, wherein each of the thermally active actuators comprise a conductive coiled fiber configured to move from the contracted state to the extended state in the absence of power.

4. The artificial muscle construct device of 1, wherein the thermally active actuators are interconnected to form a sheath that has a shape configured to extend about at least a portion of the organ.

5. The artificial muscle construct device of 1, wherein the thermally active actuators are interconnected to form a pouch that has at least one opening configured to receive at least a portion of the organ.

6. The artificial muscle construct device of 1, wherein the thermally active actuators are interconnected to form a patch that is configured to be placed against the organ.

7. The artificial muscle construct device of 1, wherein the insulating layer is positioned on an organ-facing surface of the thermally active actuators, wherein the thermally active actuators are spaced apart from the organ.

8. The artificial muscle construct device of 1, wherein the thermally active actuators are encased in the insulating layer.

9. The artificial muscle construct device of claim 1, wherein the controller is configured to selectively communicate with the thermally active actuators in order to control individual thermally active actuators.

10. The artificial muscle construct device of 1, further comprising non-active fibers connected to at least a portion of the thermally active actuators to provide support thereto.

11. An implantable cardiac device configured to be in contact with an exterior surface of a heart, the implantable cardiac device comprising:
electrothermally active actuators interconnected to form an implantable medical device, each of the electrothermally active actuators being configured to move axially between a contracted state and an extended state;
a controller in communication with the electrothermally active actuators to control their movement between the contracted state and the extended state;
a power supply configured to supply power to the controller and the electrothermally active actuators; and
wherein at least a portion of the electrothermally active actuator being configured to impart or remove a force onto the heart.

12. The implantable cardiac device of claim 11, wherein each of the electrothermally active actuators comprise a conductive coiled fiber configured to move from the extended state to the contracted state when powered.

13. The implantable cardiac device of claim 11, wherein each of the electrothermally active actuators comprise a conductive coiled fiber configured to move from the contracted state to the extended state in the absence of power.

14. The implantable cardiac device of 11, wherein the electrothermally active actuators are interconnected to form a sheath that has a shape configured to extend about at least a portion of the heart.

15. The implantable cardiac device of 11, wherein the electrothermally active actuators are interconnected to form a pouch that has at least one opening configured to receive at least a portion of the heart.

16. The implantable cardiac device of 11, wherein the electrothermally active actuators are interconnected to form a patch that is configured to be placed against a portion of the heart.

17. The implantable cardiac device of 11, further comprising an insulating layer positioned on an organ-facing surface of the electrothermally active actuators, wherein the electrothermally active actuators are spaced apart from the heart.

18. The implantable cardiac device of 11, further comprising an insulating layer encasing the electrothermally active actuators.

19. The implantable cardiac device of claim 11, wherein the controller is configured to selectively communicate with the electrothermally active actuators in order to control individual electrothermally active actuators.

20. The implantable cardiac device of 11, further comprising non-active fibers connected to at least a portion of the thermally active actuators to provide support thereto.
